(19) United States
(12) Patent Application Publication
(10) Pub. No.: US 2025/0205515 A1
(43) Pub. Date: Jun. 26, 2025

(54) MCO PLANNING OF TREATMENTS WITH AVAILABLE TECHNOLOGIES IN RADIOTHERAPY (RT)

(71) Applicant: FRAUNHOFER-GESELLSCHAFT ZUR FOERDERUNG DER ANGEWANDTEN FORSCHUNG E.V., Muenchen (DE)

(72) Inventors: Michael BORTZ, Kaiserslautern (DE); Karl-Heinz KUEFER, Weilerbach (DE); Philipp SUESS, Budenheim (DE); Katrin TEICHERT, Kaiserslautern (DE)

(21) Appl. No.: 18/694,327

(22) PCT Filed: Sep. 26, 2022
(86) PCT No.: PCT/IB2022/059141

§ 371 Date: Oct. 23, 2024

(30) Foreign Application Priority Data
Sep. 25, 2021 (DE) 10 2021 124 814.0

(51) Int. Cl. A61N 5/10

(52) U.S. Cl. CPC A61N 5/1031; A61N 5/1038

(57) ABSTRACT

The subject of this disclosure is a method of multi-criteria optimization (MCO): in particular, the designing or fashioning of a therapy as a treatment plan for a person (P) with a treatable disease who is being treated with non-simultaneous fractionated doses of the at least two radiation devices (100, 200), wherein the designed or fashioned treatment plan defines a plurality of technical settings that are adjusted on the radiation devices prior to the treatment. This is displayed on the display device (10) for the at least two technologies (A, B) for each Pareto frontier (601, 701) over at least two criteria (c1, c2) in each instance, and this in order to form a combined Pareto frontier (801) from a field (F, F′) of the two criteria (c1, c2), arising from convex combinations of at least grid points of the first and second Pareto frontiers (601, 701); and with the formation of an area section as the field (F, F′), so that a route of limited length is formed from the field, forming the combined Pareto frontier (801), is used for interactive navigation of a mixing ratio of at least two technologies (A, B) and for planning, for a point on the combined Pareto frontier (801).

The subject of this disclosure (and its claims) is a method of multi-objective optimization (MCO).

A cancer patient is to receive radiation therapy (RT). This involves a minimum dose for the tumor tissue and maximum doses for surrounding healthy tissue structures, which should ideally not be exhausted. In principle, there are multiple technical options (technologies) with which such a treatment can be carried out for such a patient.

Examples include the following:
1. Protons
2. Photons per rotation therapy with 1 rotation
3. Photons per rotation therapy with 2 rotations
4. Photons per IMRT with 9 fields (with 9 fixed angles)
5. Photons per IMRT with 9 fields (with angles other than those in option 4)
6. State-of-the-art technology devices
7. Technically older therapy devices that have been in use for some time

Each of these technologies can be performed on one or more different therapy devices. Each device is addressed differently and requires specific planning, whereby the individual planning objectives remain identical across all of the technologies used.

Relevant from a clinical standpoint is the planning of all treatment cases (overall planning), taking into account all available therapy devices. Besides taking into account the individual medical aspects of a therapy, the overall planning also needs to schedule the treatments.

Mathematically, a multi-criteria planning problem arises with the following objectives: high probability of successful treatment while avoiding side effects with each individual therapy and making the most effective use of the available therapy devices, with which as many patients as possible can receive therapy without having to endure long waiting times (time to treatment).

The state of the art is a two-stage decision-making approach. To begin with, the technology is defined, and then this technology is planned. Multi-criteria planning methods can be used in the second step. Doctors decide on the choice of technology based on experience and the results of clinical studies. In order to decide which technology is best suited to a treatment case, it is necessary to undertake separate planning for each possible technology, compare the respective plans, and then select the “best” option.

The clinical procedure also needs to be considered: In urgent cases, treatments already scheduled on certain devices will have to be postponed or even rescheduled on other devices, or else the patient in urgent need of treatment will have to wait.

Each technology must be implemented on specific, dedicated devices—treatment with protons cannot be performed using a photon accelerator. The number of devices available for treatment is one of the limiting factors in hospitals. Treating more patients—proton systems in particular are only available to a very limited extent due to their cost and complexity-automatically leads to waiting times or rescheduling of therapy sessions. A multi-criteria optimization problem becomes apparent: treating as many patients as possible in a given time-or with a given, usually limited amount of investment capital.

If a hospital now has at least two devices that can be used for treatment, they can now plan, on a purely organizational basis (by scheduling), which (daily) fractions of a fractionated treatment of a patient are to be carried out on which of the radiation devices mentioned in the example. If one of the devices is already occupied by another patient over a period of time, this device cannot be included in the planning for the new patient. The patient must either wait a considerable amount of time or be turned away. Especially when treating malignant tumors, it is particularly undesirable to keep patients waiting or even turn them away with the excuse that the devices are occupied. It is particularly important here to start the treatment planned for the fractionation period as quickly as possible. Treating a malignant tumor, for example, is not something that can be put off for long. Such patients at particularly high risk, with the risk here being the period of time until treatment begins.

Another difficulty is when technical radiation equipment which was manufactured more recently is used in the same way as older technical radiation equipment. It turns out that the state-of-the-art, recently purchased devices are permanently in use or permanently occupied, and very few patients, if any, are willing to be treated on the older devices-if their doctors' orders even allow for this.

The technical problem of the invention lies in the ability to individually provide each patient with good therapy, whereby the quality of the therapy also includes minimizing the waiting time of each patient (under “time to treatment,” a technical criterion of therapy planning).

The invention is based on the realization that a planned total dose of a planned therapy is spread over multiple treatment sessions and that the fractions originate from radiation devices that are not identical. It is assumed that two radiation devices are not the same and therefore that some daily doses are delivered by one radiation device and other daily doses by another radiation device. Taking into consideration the therapy as a whole, this results in a combinational effect through a sum of the doses per day over the duration of therapy. When a “mixture of technologies,” “mixed therapy,” or “combined radiation emission” is referred to, this means that a certain number of (daily) fractions of the planned therapy (with a total number of fractions) are administered on one device and the remaining number of (daily) fractions on another device. However, this is all part of the planning of this therapy, not the therapy itself. References to this always involve the planning.

If two radiation devices—therapy device A and therapy device B—are used, there is a solution in which only therapy device A emits its beams onto the patient, in which case it would be a pure A treatment. There is another version in which only therapy device B emits its beams onto the patient, in which case it would be a pure B treatment.

Any other type of combined radiation emission from both devices to the same patient during the therapy period is combinational irradiation, which lies between “A only” and “B only” in its effect. This involves the planning of these therapies, not their use.

Reference is made to the claims, which are included here for solving the problem mentioned, in particular independent claims 1 and 7.

Embodiment examples of the invention are explained in more detail with the aid of the Figures. All explanations are equally applicable to the disclosure, but they are not to be interpreted in such a way that they must be included as necessary elements of the claims. All of the following examples remain examples even if they are not explicitly preceded by “for example.”

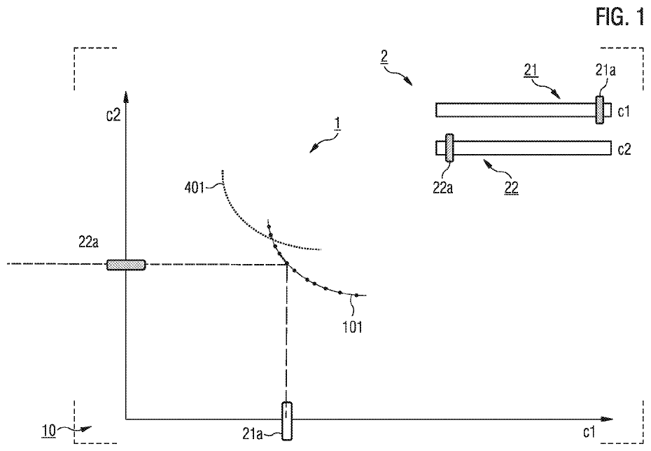

FIG. 1 is a schematic view of a sample sequence of a process for planning a therapy. Here with patches 401 and 201 as technologies on a display device with display 10 (represented by the four dashed corners) and two operating aids 21, 22 as sliders with respective “handles” 21a and 21b (as graphically depicted control buttons). Patch 101 is shown as patch 201 in the following FIG. 1A with its grid points, which here apply to patch 101. Patch 201 corresponds to patch 101.

FIG. 1A helps with understanding the Pareto frontier—in the example of a Pareto frontier 201. A large number of grid points 201₁ to 201₁₀ can be seen. This is to be transferred to Pareto frontier 101 of FIG. 1, where it applies as corresponding grid points 101₁ to 101₁₀. Therefore, frontier 101 is by no means continuous, but rather only functionally connected, which is why it is also shown as a (thin) line in FIG. 1, which is not really a line. The Pareto frontier can also be thought of as a projection on the n-dimensional space in which it exists, whereas it can only be visualized as a projection.

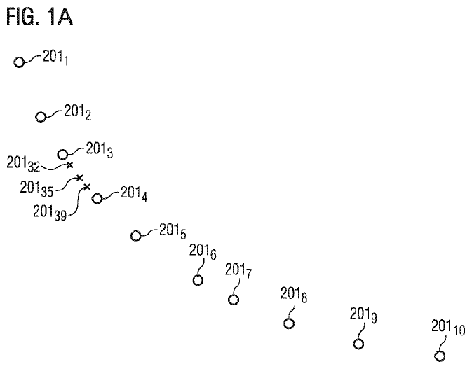

FIGS. 2A to 2D are different radiation devices 100, 200 and 300, 300′, which provide different radiation devices (or “technologies”). These include both state-of-the-art devices with new technology and devices that have been in use for some time. According to FIGS. 2C and 2D, there may be a device A of technology 300 and another device B of technology 300′ that is already older and is therefore less likely to be selected by the treating physician for his/her planning if no other criteria play a role, such as immediate availability of the radiation device in order to start planning first with the radiation device according to FIG. 2D (thereby preparing for later therapy). This is the case if radiation device A is occupied and therefore not available. The radiation device of older technology 300′ in FIG. 2D is to be interpreted as a “different technology,” even though it would technically be the same technology as the modern radiation device of FIG. 2C with technology 300.

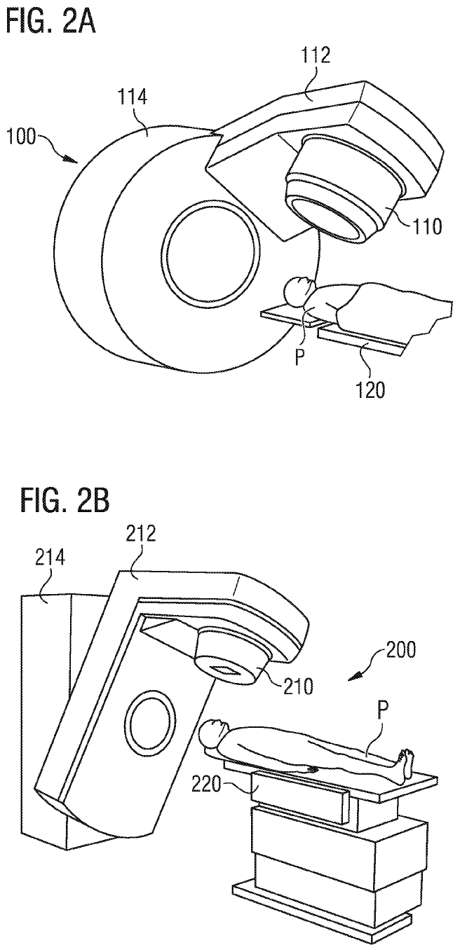

FIGS. 4A to 4D show a sequence where a field F arises or is defined which maps all combinations of achievable mixing ratios of two technologies with Pareto frontiers 601 and 701—and this is with target criteria c1 and c2.

FIG. 4E illustrates an alternative formation of field F using the same points on the Pareto frontiers as in FIG. 4B.

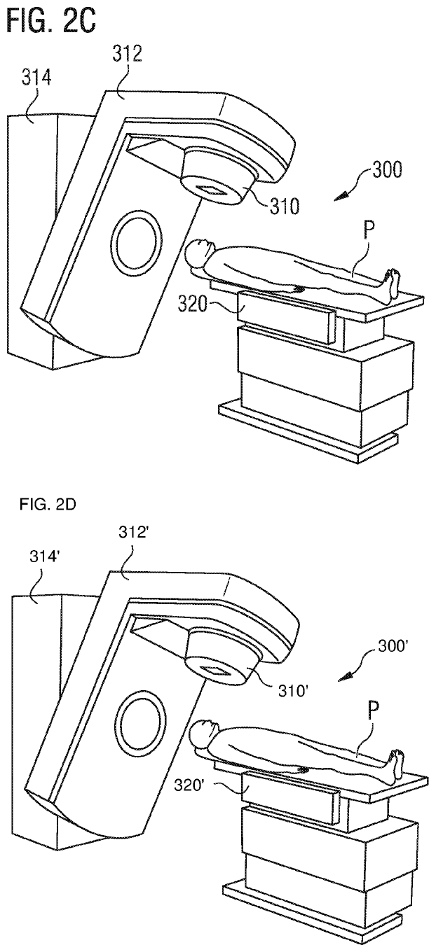

FIGS. 5A and 5B show a limitation of achievable values in target criteria c1 and c2 by intervals I₁′ and I₂′ (also called therapeutic window f), whereby the space of achievable mixing ratios of technologies is restricted, shown here as restricted field F′.

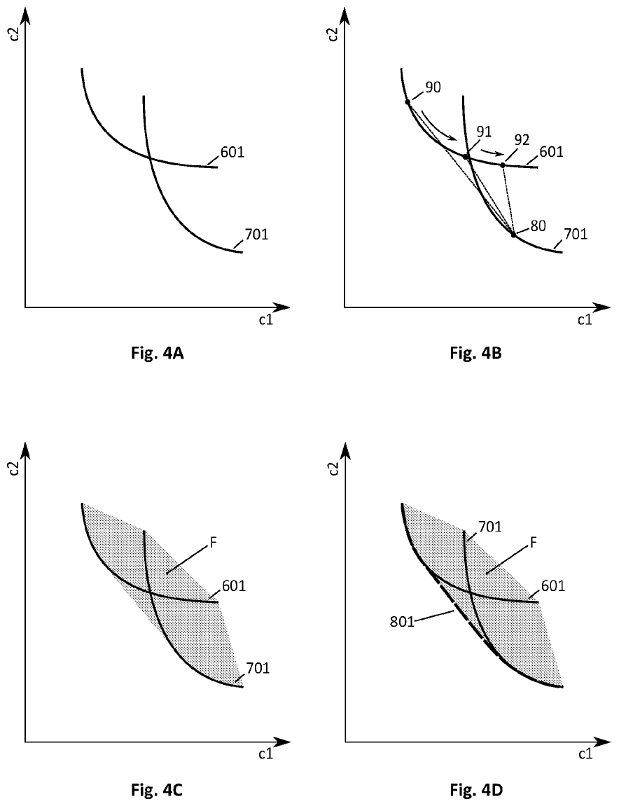

FIG. 6 shows a further restriction of field F′, thereby creating fields F₁″ and F₂″. The further limitation results from setting a predefined minimum (or maximum) proportion of fractionations per technology. The criterion “at least a ⅔ proportion of technology 601” (or at most a ⅓ proportion of technology 701) is shown.

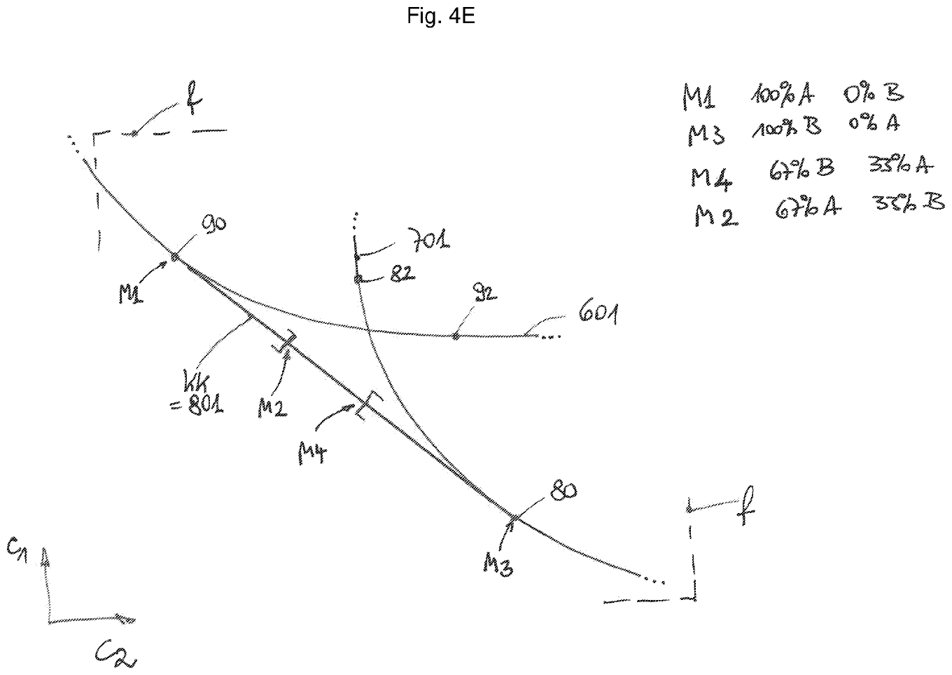

FIG. 7 shows field F′ in the event that one technology is strictly better than another technology.

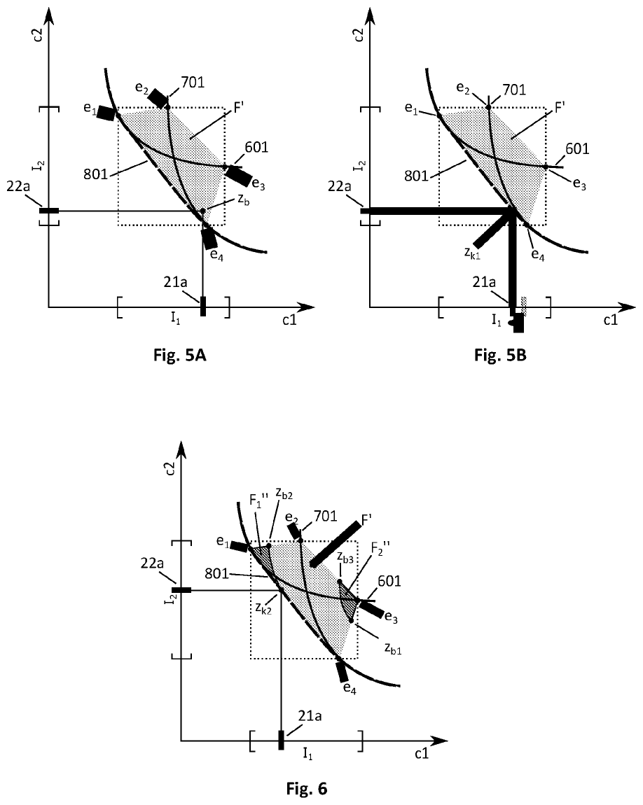

FIG. 8 shows field F′ in the event that there is a gap 1401 in the value range of one of the target criteria for two selected technologies.

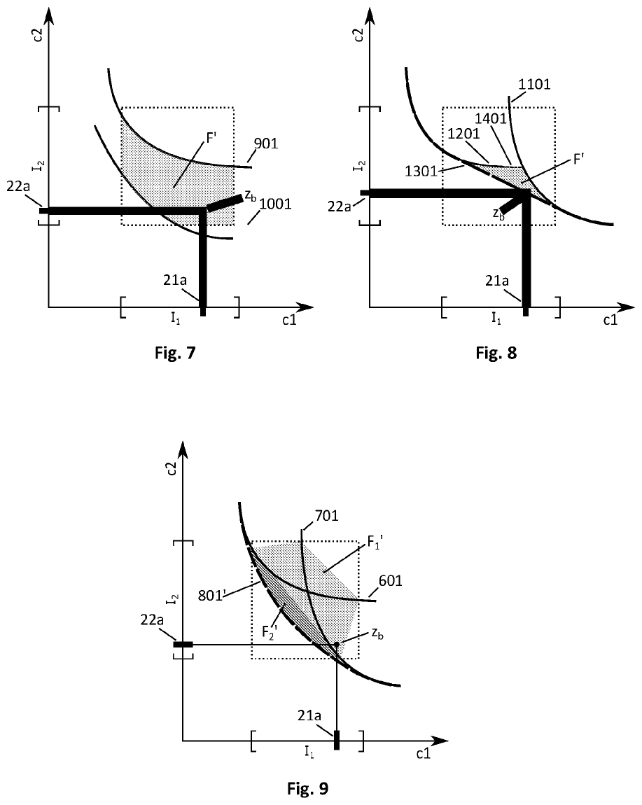

FIG. 9 shows field F′, here consisting of F₁' and F₂′, in the event that synergy effects result from the combination of two technologies. Field F′ is extended.

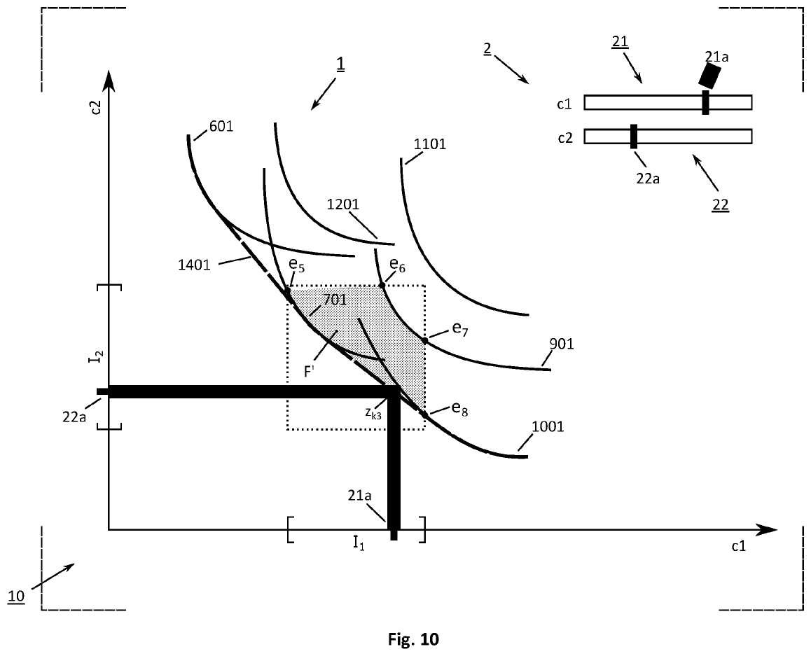

FIG. 10 shows the Pareto frontiers (with their invisible grid points interpolated between them; see FIG. 1A). The shapes of Pareto frontiers 601 to 1201 can be seen from FIGS. 4A to 9. Point zₖ₃, currently selected via the sliders, is determined by sliders 21a and 22a in control area 2 of the display, whereby 1 forms the functionally separated patch area of the Pareto frontiers.

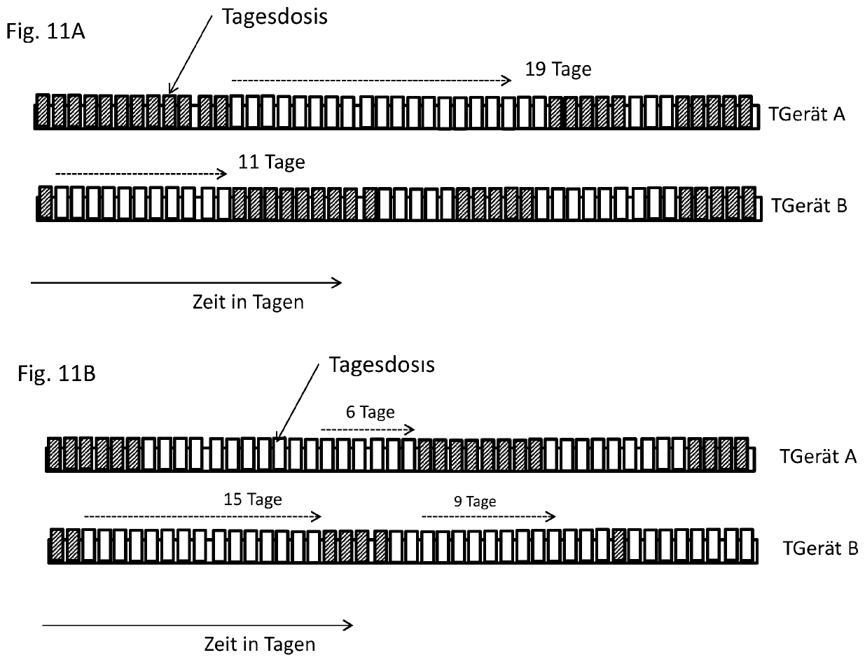

FIGS. 11A/11B illustrate the temporal availabilities of therapy devices A and B (referred to as “T devices”) in a daily grid of availabilities (light is free, shaded is occupied).

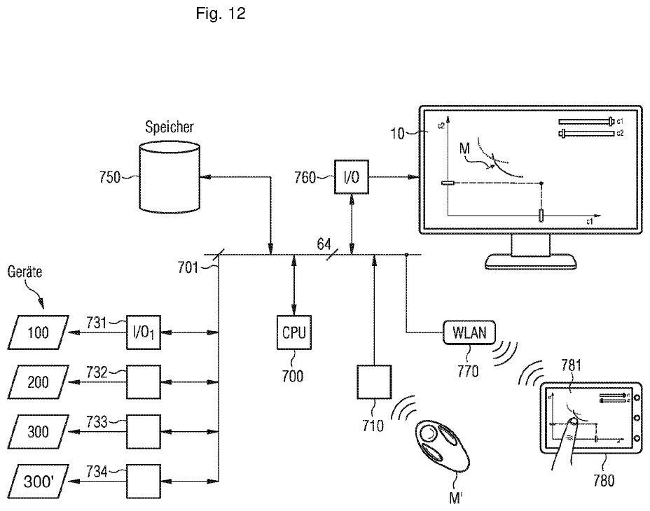

FIG. 12 illustrates a technical-functional data system with a computer, memory system, and input/output device with a display 10. Display 10 can be a normal display device or a touch screen giving navigating control over the planning system with a finger (as a pointing device) instead of a mouse pointer.

The description deals with the fact that not only one therapy device (as a radiation device) is available, but several of them. Naturally, the therapy devices have an occupancy rate, i.e., their occupancy is scheduled with existing therapies in a time grid, so that a new patient needing therapy has to fit into the existing system of occupancy.

It leads to resentment and even fear if a patient with a critical case of cancer requiring treatment cannot be treated immediately, i.e., no treatment slot is available and no treatment can be planned for them. In other words, no radiation device is available at the moment when the patient announces his or her need for treatment or when it is assigned by the planner.

One way of making this earliest possible treatment available through its underlying planning is to incorporate a different radiation device or a radiation device of another technology into the treatment plan—a radiation device of an older technology is also possible. This results in a mixture of multiple daily doses from different radiation devices.

Here, “mixture” is not intended to mean that the radiation devices are mixed, but rather that the effects of the radiation devices on the patient requiring therapy are mixed (or combined with daily doses over the course of the therapy).

Again, it bears repeating that no therapy is currently being performed on humans (the patient) here, but is only planned. This planning can already be implemented outside the patent claims as therapy with the radiation devices, not with the patent claims. Nevertheless, when describing the planning, it is sometimes necessary to mention radiation therapy per se.

Below, radiation device A and radiation device B—based on the figures, it is their Pareto functions 601 and 701—are to be considered as the at least two radiation devices whose effects in the planning (for the duration of the fractionated therapy) act together on the patient requiring therapy (correct: “to be assumed to act”). Multiple radiation devices of this kind are possible and able to be integrated into the planning.

For this to be possible, the mixture occurs in fractionated daily doses. The period is considered to be the entire duration of treatment, and the smallest unit of the mixture is one day. This division of the units, the total duration and, discretely, the day are to be understood as an example. It is based on the current standard fractionated planning for a patient, where one day has proved to be a good, suitable unit in which a patient is able to bear function, recovers, and is able to function again.

Therapy planning begins on day 1 and ends after the duration of treatment on day X, where X is given as 30 in the examples.

After a day Y<X, the planning switches to another radiation device. We start with the first radiation device.

The premise for the first device should be “start therapy as soon as possible.” The patient requiring treatment therefore has no appreciable waiting time and has the feeling that he or she can be treated right away. In most cases, the technically best possible radiation device or the radiation device with the most state-of-the-art technology is not immediately available, so the waiting time for the patient requiring treatment seems disruptive, unpleasant, or even frightening. In contrast, offering a therapy that starts immediately is preferrable, even if the immediately available device is not state-of-the-art or does not offer the most technically advanced technology, this radiation device should still be scheduled at the beginning. Scheduling could be used as an input value, but since the quality of treatment also plays a role, mere scheduling is not sufficient.

A second premise, although not a mandatory one, is to reduce the number of changes between radiation devices. It will be perceived as unpleasant by the patient if—according to the planning—he or she has to or is supposed to change the radiation device multiple times, i.e., if the planning is such that he or she is made aware of the availability. On the other hand, it is divided into daily doses, and after a day passes, a patient will no longer remember exactly on which radiation machine he or she received the fractionated daily dose the day before.

What is decisive is the dose distribution in the relevant voxels of the tissue over the entire period of the planned therapy, described here as the therapy to be planned, since only this planning is described here and is also claimed, not the radiation therapy itself that is carried out on the patient.

The criteria, ci, according to which a therapy is planned are of a technical nature—for example, selected from the following “mean dose in the heart,” specific clinical objectives, dose in the target, or duration. The technical question that arises is “what quality of treatment is possible” under the premise of “the earliest possible start.” Mathematically, this involves a mixed-integer, non-convex problem that only works with the non-dominating points (a point on the Pareto frontier corresponds to a plan with its technical setting parameters).

The premise of the planned time to treatment (TTT) sets the framework for the mixing ratio for the total duration of therapy planning.

If there are multiple changes between the radiation devices, meaning planned changes, this will have a restrictive effect on the mixing ratio over the total duration.

Quantity F of the efficient mixtures is regarded as being within the tolerable limits. There is both an upper limit and a lower limit defining a minimum radiation dose. To this end, a therapeutic window, labeled ‘f’ in the figures, is opened; see FIG. 4E, for example. The therapeutic window can change dynamically or vary in size.

FIG. 1, enlarged, shows an operating area 2 at the top right and, taking up more space than operating area 2, a patch area 1 on the left. Operating area 2 is outside patch area 1, which means that these two do not overlap in a functionally obstructive manner. They appear visibly separated from each other for the planner, whereby their position is only selected in the example in FIG. 1 in such a way that the patch area is arranged at the bottom left and the operating area at the top right (on the visible surface of display 10). Other arrangements are also possible.

Operating area 2 and patch area 1 are functionally linked to one another. This functional coupling bears explaining.

Several Pareto frontiers are shown in patch area 1; in the example, these are Pareto frontiers 101 and 401 along with 601, 701, and so on in FIG. 4A to FIG. 10.

Also shown in patch area 1 are two axes c1 and c2 (criterion 1 and criterion 2), which are perpendicular to each other and represent two criteria c1 and c2. Criterion 1 (c1) and criterion 2 (c2) are shown, which are visible and navigable for the planner in operating area 2. The limitation of the illustration to two criteria here is only by way of an example; many more criteria than these will be factored in. Examples of possible technical criteria are listed above. However, since only three criteria can be visibly displayed in a representable Cartesian space, not n criteria where n is greater than 3, we will leave this example as is and merely state that n criteria are factored in, i.e., c1 to cn. The illustration can also be a projection from n-dimensional space.

The following should be noted for an understanding of the Pareto frontier(s). Each Pareto frontier is a function that is only functionally connected. Each of them has a large number of grid points (here, 101; instead of 201; as shown in FIG. 1A). The frontier is therefore by no means to be understood as continuous, but only as “functionally connected;” therefore, a line is also shown in FIG. 1 for the grid points, which basically does not show a continuous Pareto frontier. It can also be a projection from the n-dimensional space, in which space it exists, whereas it can only be shown graphically as a projection. The lines represent the functionally connected Pareto frontier. This is illustrated in enlarged form in FIG. 1A, with interpolation between the grid points.

The first radiation device 100 of FIG. 2A is a radiation device 100 for emitting protons during or for radiation therapy to a patient P positioned on a table 120 (ready for treatment).

The first radiation device stands for technology 100 (or technology A). A first support body 114 is rotatably mounted, and a radiation head 110 is arranged on it, which is rigidly connected to support body 114 via a bridge 112. The angle of radiation head 110 relative to patient P can be adjusted via support body 114. The radiation dose and the distribution of the radiation within the proton beam from radiation head 110, which is not shown, can also be adjusted. All these technical values are represented in a Pareto frontier for a patient over the entire course of a fractional radiotherapy (radiation therapy). In the example, this can correspond to Pareto frontier 101 of FIG. 1. The technical values as setting variables of radiation head 110 are set via an I/O interface 731 from 64-bit bus 701, for example, which can be seen in FIG. 12.

It should again be pointed out that it is not the therapy with radiation device 100 (technology 100) that is claimed here, but rather the planning of this therapy. The reference to the therapy devices as radiation devices, which implement this planning later or functionally separately from the planning, serves as an illustration for the patient, but the disclosure is not intended to refer to the patient's therapy. Planning and therapy can be readily separated or differentiated in terms of time and function.

The same applies to the following figures.

FIG. 2B shows another radiation device, here a photon radiation device 200 of technology 200 (or technology B). Patient P is placed on a table 220, and radiation head 210 for emitting the protons is arranged on an L-shaped bridge 212. Bridge 212 is rotationally connected to a stationary base 214, relative to which it can be pivoted. Here, too, the settings of the beam emission of radiation head 210 can be prescribed, and they can be prescribed or set for a fractionated treatment of a Pareto frontier, such as Pareto frontier 101 of FIG. 1. The technical values of radiation head 210 are set via an I/O interface 732 from 64-bit bus 701, for example, which can be seen in FIG. 12.

FIG. 2C shows another radiation device 300 of technology 300. Here, too, patient P is laid out on a table 320. A bridge 312 is arranged so that it can pivot on a base 314, and radiation head 310 is designed to emit accelerated photons. The emission of the accelerated photons from the beam head 310 is adjusted so that they are matched to the target and the risks in such a way that the target is primarily covered by the beams in its volume during the fractionated treatment session and the risks are shielded from exposure to radiation. To this end, a multi-slat collimator—not shown—can be provided, which is placed in radiation head 310 and whose slats are adjusted in such a way that they are brought into any desired shape in order to release a suitable area as free space between them, corresponding to the target volume in the best possible way. The slats themselves are beam-shielding—made of tungsten, for example—so that the shape of the beam can be adjusted almost at will. In addition to this shaping adjustment of the radiation volume (the radiation area, actually), a regimen can be used to specify the fields and angles at which the irradiation occurs. The technical values of radiation head 310, such as the multi-slat collimator, are set via an I/O interface 733 from aforementioned 64-bit bus 701, for example, which can be seen in FIG. 12.

FIG. 2D shows another radiation device 300′, which corresponds to that of FIG. 2C, but is an older or technically old design, which can correspond to therapy device D of the following description. The older design is intended to express the fact that it is a separate technology 300′ that corresponds to that of the therapy device in FIG. 2C but does not correspond to the current state of technical possibilities. The components used here correspond to those in FIG. 2C. They are symbolically marked with an apostrophe, i.e., (old) bridge 312′, (old) base 314′ and (old) radiation head 310′. Patient P is placed on (old) table 320′. “Old” stands for older technology 300′. The technical values of technically older radiation head 310 are set via an I/O interface 734 from aforementioned 64-bit bus 701, for example, which can be seen in FIG. 12.

In the sequence of FIGS. 4A to 4D, a field F arises that maps all combinations of achievable mixing ratios of two technologies with Pareto frontiers 601 and 701—and this is with target criteria c1 and c2.

FIG. 4E shows how field F arises. Two lines follow Pareto frontiers 601 and 701. A line 80-90 is fixed at point 80 and moves from point 90 on Pareto frontier 601 to point 92. The area covered circumscribes a first group of points belonging to field F, which is not shown here. The second line also extends between points 90 and 80, whereby this line is fixed at point 90 and runs along Pareto frontier 701 to point 82 (see FIG. 4E). This is the second group of points of field F. Together they form field F. Its lower left edge is the sum of the Pareto-optimal points whose connection results in line KK or 801, the convex combination of points.

Along line 801 of the Pareto-optimal points, the mixing ratio can be adjusted, i.e., changed in a navigable manner (during planning). Two points are highlighted, which result from the possible mixing ratio of FIGS. 11A and 11B due to the availability of the radiation devices.

This is point M2 marked by a square bracket with approx. 67% of Pareto frontier 601, which here stands for radiation device A. In contrast, point M4 marked by an oppositely aligned square bracket has a proportion of approx. 67% of radiation device B. Accordingly, a proportion of approx. 33% remains in the mixture for the respective other radiation device.

As the points to the right of M4 are closer to Pareto frontier 701, the proportion of Pareto frontier 701 is greater here. Point M3 has a 100% share of radiation device B. Point M1 is located on Pareto frontier 601 and therefore has a 100% share of radiation device A. A point corresponds to a therapy plan with its technical settings on the radiation device.

The two points M2 and M4 yielded from the availability of the therapy devices, here with a view to FIGS. 11A/11B. FIG. 11A shows the two radiation devices T-Device A and T-Device B with their respective time slots of daily doses. The premise of starting as quickly as possible means that radiation device A cannot be scheduled. Only radiation device T-Device B is available as quickly as possible. For planning purposes, there is availability for eleven daily doses before this radiation device is also occupied. It will then be possible to switch to radiation device A. The remainder of the 30 daily fractions is scheduled there with 19 daily doses. This results in a distribution of approx. one third of radiation device B and approx. two thirds of radiation device A. This results in the two “points” M2 and M4 marked by square brackets on path KK or 801 in FIG. 4E.

In a second example in FIG. 11B, radiation device A is initially occupied for five days, which means that it cannot provide the fastest possible start of the planned therapy. Radiation unit B is initially only occupied for two days and is then available for 15 days in the planning. However, the switch to therapy device A that is then to be planned is only possible for the following six days, with a switch back to therapy device B that is subsequently to be planned for the remaining nine days of the planning period of 30 days also assumed here for the entire (duration of) fractionated therapy.

The switching back of the therapy device may make medical sense, but is not actually desirable as a first choice. It could make sense from a medical standpoint for two reasons:
a. It protects a specific organ Z1.
b. It puts strain on another organ Z2 that can cope with the strain.

It should be mentioned here that point M1 depicted is dominated by the Pareto frontier 601 with regard to criterion c2.

FIG. 11B shows very clearly that the goal of planning can also be to fill gaps in the availability of radiation devices, provided that this makes medical sense.

FIGS. 5A and 5B show that navigation along line 801 (the sum of the Pareto-optimal points) is also possible using sliders 21a and 22a of operating aids 21 and 22 shown in FIG. 1.

FIG. 7 illustrates a field F′ between two technologies 901 and 1001, where one of the technologies is strictly better than the other. Therapies that are closer to function B as a representative of radiation device B can be started earlier than the planning of therapies that are closer to function A.

Another possibility for the position of the Pareto frontiers of radiation devices A and B, corresponding to 601 and 701, is for there to be a gap 1401 in the therapeutic window of criterion c1, which can be bridged by line 1301 (in the planning) with a mixing ratio of the two therapies that are to be planned.

If synergy effects result from FIG. 9, line 801′ adds a further bulbous section F2′ to common area F (in addition to existing area F1′).

FIG. 12 shows a network-orientated representation of the control system via aforementioned bus 701 and the digital components used that are coupled to it. In the preferred example, bus 701 is a 64-bit bus via which the digital components communicate with one another. It is bidirectional. For the computing capacity and computing function, CPU 700 is specified, which reads functions 401 and 101 displayed on display 10 from memory 750 and forwards them to display unit I/O 760 via bus 701. Depending on the technical type of display 10, the data is processed and visualized in display unit 760.

The navigation environment of FIG. 1 is shown. It is operated via function pointer M and moved and activated by the planner via a mouse device, shown here in the example as trackball M′ (selection of a function at a location of pointer M). Trackball M′ is coupled by radio to a receiver 710, which converts its movement signals and forwards them to bus 701. This coupling is not bidirectional.

Mouse device M′ is assigned to display 10, e.g., as the trackball, which enables the functions of pointing and operating (triggering a function) at the location where mouse pointer M is displayed.

Mouse pointer M is also used to operate both operating aids 21 and 22 for criteria c1 and c2.

An alternative display 781 is that of a tablet 780, which is given the same representation of FIG. 1, which is shown on touch-sensitive display 781. As explained above, the screen display or tablet display has a patch area 1 and operating area 2. Tablet 780 is coupled to bus 701 via a Wi-Fi coupling (or connection) with a Wi-Fi transmitter and receiver 770. The mouse pointer is replaced by a finger (as shown) and controlled by gestures (e.g., swipe, tap, double tap) on display 781 of screen-enabled or touch-sensitive tablet 780.

Technological radiation devices 100 to 300′ are each coupled to bus 701 via one input/output device 731, 732, 733 and 734, respectively. Each of these input/output devices is bidirectional, meaning that it can preset parameters from bus 701 to the respective radiation device.

Preferably, each of these radiation devices 100, 200, . . . has sufficient memory of at least 500 GB so that it can store the preset parameters of the subsequent therapy and set them independently, i.e., autonomously, during the therapy, especially for fractionated sessions of the patient during the planning period.

In the example, I/O₁ sends the setting parameters to radiation device 100 for the emission of protons. This is then able to apply the parameters of the radiation device for technology 100 “protons” that are to be set—which are actually already prescribed—during the (fractionated) therapy of patient P spread over days. In other words, the doses and intensities and directions of proton radiation (in radiotherapy) to which this individual patient P is to be exposed. This is where the functional separation of planning and therapy occurs.

This example can also be controlled in such a way that the parameters are stored in memory 750 for the duration of the fractionated therapy and are only transmitted to radiation device 100 at the times before the respective therapy session. This fractionated therapy is fractionated programming of respective radiation device 100 to 300′ (akin to the fractionated data transmission of the currently required time section of the therapy, such as 30 days). The actual therapy is performed automatically by radiation device 100, without the previously completed planning.

In the same way, the other devices 200, 300 and 300′ are programmed, prepared for therapy,: and conditioned with regard to data technology.

It bears emphasizing once again that no therapy is performed during the planning stage; the therapy is already fully planned and functionally prepared (i.e., fully planned) before the respective device performs this radiotherapy on the patient. The latter therapy is not claimed.

Navigation via the patches shown on display 10 can only be completed by the planner in order to transfer them later to the associated radiation devices via respective input/output devices 731 to 734.

1. Procedure for designing or fashioning a therapy as a treatment plan, before treatment, and with interactive navigation on a display device (10)
(a) wherein at least two technologies (A, B) of at least two radiation devices (100, 200, 300, 300′) are displayed on a display device (10) and are available for selection, with at least one technology (A) of a first radiation device (100) and at least one technology (B) of a second radiation device (200) being provided;
(b) after the designing or fashioning, for a person (P) with a treatable disease who is being treated with non-simultaneous fractionated doses of the at least two radiation devices (100, 200), wherein the designed or fashioned treatment plan defines a plurality of technical settings that are adjusted on the radiation devices prior to the treatment;
(c1) wherein a Pareto frontier (601, 701) is displayed on the display device (10) for each of the at least two technologies (A, B) using at least two criteria (c1, c2);
(c2) forming a combined Pareto frontier (801) from a field (F,F′) of the two criteria (c1,c2), created from convex combinations of at least grid points of the first and second Pareto frontier (601,701);
(c3) forming an area section as a field (F, F′);
so that a line of limited length is formed from the field, which forms the combined Pareto frontier (801), is made available for interactive navigation of a mixing ratio of at least two technologies (A, B) and is used for planning, designing, or fashioning the treatment plan as a point on the combined Pareto frontier (801).

2. Procedure according to claim 1, wherein interpolated intermediate values of the grid points are also mapped by the convex combination in new points next to the Pareto frontiers.

3. Procedure according to claim 1 or 2, wherein the line as a combined Pareto frontier (801) does not intersect the Pareto frontiers (601, 701).

4. Procedure according to one of the previous claims, wherein the new points formed by convex combinations next to the Pareto frontiers (601, 701) span the field (F, F′) in the therapeutic window (f).

5. Procedure according to any one of the preceding claims, wherein the planning point on the combined Pareto frontier (801) comprises fractionated portions of the technology (A) of the first radiation device (100) and fractionated portions of the technology (B) of a second radiation device (200).

6. Procedure according to both of the preceding claims, wherein sets of points are taken together to form an area section, and these are placed in a ratio that is limited by temporal availability of the at least two radiation devices (100, 200, 300, 300′).

7. Display device (10) for designing or fashioning a therapy as a treatment plan, before treatment, and with interactive navigation on the display device (10).

8. A display device according to claim 7, designed for designing or fashioning according to any of the preceding claims 1 to 6.
